(19) United States
(12) Patent Application Publication
(10) Pub. No.: US 2009/0036549 A1
(43) Pub. Date: Feb. 5, 2009

(54) SUBSTANCE-ENCAPSULATED CARBON NANOHORN COMPLEX AND PRODUCING METHOD THEREOF

(75) Inventors: Ryota Yuge, Tokyo (JP); Masako Yudasaka, Tokyo (JP); Sumio Iijima, Tokyo (JP); Hideki Yorimitsu, Kyoto (JP)

Correspondence Address: SCULLY SCOTT MURPHY & PRESSER, PC, 400 GARDEN CITY PLAZA, SUITE 300, GARDEN CITY, NY, 11530, US

(73) Assignees: NEC CORPORATION, Tokyo (JP); KYOTO UNIVERSITY, Kyoto (unknown)

(21) Appl. No.: 12/278,111

(22) PCT Filed: Feb. 2, 2007
(86) PCT No.: PCT/JP2007/052291

§ 371 Date: Aug. 1, 2008

(30) Foreign Application Priority Data
Feb. 6, 2006 (JP) 2006-027935

(51) Int. Cl. A61K 47/30; A61P 43/00

(52) U.S. Cl. 514/772.1

(57) ABSTRACT

A cap of polyamine molecules is provided on an aperture portion of carbon nanohorns having apertures formed by oxidation. The polyamine cap opens and closes according to the pH of the ambient environment, thereby controlling release of an encapsulation substance.

### TECHNICAL FIELD

The present invention relates to a substance-encapsulated carbon nanohorn complex, a producing method thereof, a substance release control method using a substance-encapsulated carbon nanohorn complex, and a drug delivery system medicine.

### BACKGROUND ART

In recent years, it has been examined to utilize various inorganic substances as carriers for drugs in a drug delivery system. Particularly, attention has been focused on nanoparticles in use for such carriers. There have heretofore been many reports on such nanoparticles.

In such a situation, there has been a growing interest in nanocarbon materials of nanosize, such as carbon nanotubes and carbon nanohorns. Some attempts have been made to modify those nanocarbon materials so as to generate functions such as biocompatibility and drug properties, as well as properties resulting from characteristic structures of nanosize substances.

For example, Japanese laid-open patent publication No. 2005-343885 (Patent Document 1) gives attention to unique structures and characteristics of carbon nanohorns and discloses technology relating to a novel complex and a producing method of a novel complex in which functional organic molecules having physiological activity or pharmacological activity are encapsulated and supported in carbon nanohorns.

Furthermore, Murakami et al., “Molecular Pharmaceutics,” American Chemical Society, 2004, Vol. 1, No. 6, pp. 399-405 (Non-patent Document 1) describes that the aforementioned carbon nanohorn complex encapsulating drug therein can be applied to a drug delivery system (DDS) medicine because it can realize sustained-release.

### DISCLOSURE OF INVENTION

The invention described in Patent Document 1 and the technology reported in Non-patent Document 1 allow various substances including medicine to be encapsulated in carbon nanohorns and to be released from the carbon nanohorns. However, an encapsulated substance is released by spontaneous action. Therefore, such technology has a problem that it has difficulty in practical application to a drug delivery system (DDS), which selectively releases a medicine in a body.

The present invention has been made in view of the above, and its object is to provide a carbon nanohorn complex capable of solving problems in the prior art and controlling release of an encapsulated substance.

The present invention has the following features to solve the above problems.

Specifically, in a substance-encapsulated carbon nanohorn complex, a first aspect of the present invention is characterized in that a cap of polyamine molecules is provided on an aperture portion of carbon nanohorns produced by oxidation for formation of apertures so as to selectively open and close the cap according to a pH environment.

Furthermore, in the substance-encapsulated carbon nanohorn complex according to the first aspect, a second aspect of the present invention is characterized in that an amino group of the polyamine molecules is adsorbed on a carboxyl group existing as a substituent at the aperture portion.

Moreover, in the substance-encapsulated carbon nanohorn complex according to the first or second aspect, a third aspect of the present invention is characterized in that an encapsulation substance is one of organic matter, inorganic matter, and metal, or a mixture or a compound of two or more thereof.

Furthermore, in a method of producing a substance-encapsulated carbon nanohorn complex, a fourth aspect of the present invention is characterized by encapsulating, in a solution, an encapsulation substance into carbon nanohorns having apertures formed by oxidation, and then attaching a cap of polyamine molecules in a solution that does not dissolve or is unlikely to dissolve the encapsulation substance so as to prevent the encapsulation substance from being released from an interior of the carbon nanohorns when the cap is being attached.

Moreover, in the method of producing a substance-encapsulated carbon nanohorn complex according to the fourth aspect, a fifth aspect of the present invention is characterized by cleaning the carbon nanohorns having apertures formed by oxidation after reaction with amines so as to complete an unnecessary reaction other than an electrostatic interaction in advance, and then encapsulating the encapsulation substance into the carbon nanohorns.

Furthermore, in the substance-encapsulated carbon nanohorn complex according to any one of the first to third aspects or in the substance-encapsulated carbon nanohorn complex produced by the production method according to the fourth or fifth aspect, a sixth aspect of the present invention is characterized in that the encapsulation substance encapsulated in the carbon nanohorns is eluted from an interior of the carbon nanohorns to an ambient environment so as to sustain release of the encapsulation substance when the cap of polyamine molecules opens.

Moreover, in a substance release control method using the substance-encapsulated carbon nanohorn complex according to the sixth aspect, a seventh aspect of the present invention is characterized in that a pH is set to be less than 7 so as to open the cap of polyamine molecules for eluting the substance encapsulated in the carbon nanohorns to the ambient environment and sustaining release of the substance.

Furthermore, in the substance release control method according to the seventh aspect an eighth aspect of the present invention is characterized in that a pH of the ambient environment is set to be not less than 7 so as to close the cap of polyamine molecules for stopping the elution of the substance encapsulated in the carbon nanohorns to the ambient environment.

Moreover, in a drug delivery system (DDS) medicine, a ninth aspect of the present invention is characterized by including the substance-encapsulated carbon nanohorn complex according to any one of the first to third and sixth aspects or the substance-encapsulated carbon nanohorn complex produced by the production method according to the fourth or fifth aspect.

Effects of the Invention:

According to the present invention, a cap of polyamine molecules is provided on an aperture portion formed on a surface of carbon nanohorns. Therefore, the aperture portion is selectively opened and closed accurately in response to a pH environment. As a result the release control of a substance encapsulated in the carbon nanohorns can be achieved by using a pH environment. Thus, the present invention can be applied to a DDS medicine and the like.

### BRIEF DESCRIPTION OF DRAWINGS

FIG. 1 is a table showing weight ratios of C₆₀/carbon nanohorns, which were obtained from TGA measurement on C₆₀-encapsulated carbon nanohorns produced in Example 1 and C₆₀-encapsulated carbon nanohorns having a spermine cap, and the amounts of C₆₀ released in toluene, which were estimated based on visible-ultraviolet absorption spectra, before and after addition of CF₃COOH.

FIG. 2 is a table showing weight ratios of CDDP/carbon nanohorns, which were obtained from TGA measurement on CDDP-encapsulated carbon nanohorns produced in Example 2 and CDDP-encapsulated carbon nanohorns having a TMTACTD cap, and the amounts of CDDP released in an aqueous solution, which were estimated based on visible-ultraviolet absorption spectra, before and after addition of CF₃COOH.

### BEST MODE FOR CARRYING OUT THE INVENTION

The present invention has the aforementioned features, and embodiments of the present invention will be described below.

Carbon nanohorns used as a starting substance are aggregates each having a diameter of 2 nm to 5 nm. Aggregates having a diameter of 30 nm to 150 nm can be used. Apertures are formed in carbon nanohorns by an oxidation treatment. The size of the apertures can be controlled by controlling the oxidation conditions. For example, in a case of oxidation under oxygen, the size of apertures in carbon nanohorns can be controlled by changing the oxidation treatment temperature. Apertures having a diameter of 0.3 nm to 1 nm can be formed at a temperature of 300° C. to 420° C. Furthermore, apertures can also be formed in carbon nanohorns by treatment with an acid or the like.

Means for encapsulating a substance in carbon nanohorns having apertures formed by an oxidation treatment is implemented by mixing carbon nanohorns having apertures and an encapsulation substance in a liquid phase and evaporating the solvent. It is effective to perform the evaporation of the solvent in an inert gas. Carbon nanohorns encapsulating an encapsulated substance are referred to as a substance-encapsulated carbon nanohorn complex.

The liquid-phase solvent used to mix carbon nanohorns and an encapsulation substance in a liquid phase can be selected in a suitable manner. That is, anything (any solvent) that dissolves an encapsulation substance allows the encapsulation substance to be encapsulated in carbon nanohorns.

Furthermore, an encapsulation substance to be encapsulated in carbon nanohorns can be any substance as long as it can be dissolved in a solvent and can exist in the solution. One of organic matter, inorganic matter, and metal, or a mixture or a compound of two or more thereof, or the like may also be used.

The aperture portion of the substance-encapsulated carbon nanohorn complex is provided with a cap of polyamine (molecules) having amino groups. For example, spermine, 1,1,4,7,10,10-hexamethyltriethylenetetramine, and 1,4,8,11-tetramethyl-1,4,8,11-tetraazacyclotetradecane are suitable for polyamine as a cap.

Addition of a cap to the substance-encapsulated carbon nanohorn complex having apertures is performed in a solution in which the encapsulated substance does not elute or is unlikely to elute. That is, polyamine and the substance-encapsulated carbon nanohorn complex are put and mixed in a solution that is likely to dissolve polyamine but unlikely to dissolve the encapsulated substance. Then the mixed solution is sufficiently agitated to perform the aforementioned addition. This method of adding a cap can prevent the encapsulated substance from being released from the interior of the carbon nanohorns at the time of the addition of the cap. Thereafter, the substance-encapsulated carbon nanohorn complex is separated from the mixed solution with use of a filter or the like.

Polyamine has been adsorbed as a cap on the aperture portion of the separated substance-encapsulated carbon nanohorn complex. Specifically, carboxyl groups as substituents exist at the aperture portion of the substance-encapsulated carbon nanohorn complex, and amino groups of polyamine are adsorbed on the carboxyl groups.

The carbon nanohorns having apertures formed by oxidation may be reacted in advance with amines and then cleaned with an acid or the like before the encapsulation of the encapsulation substance, for thereby performing a pretreatment to complete unnecessary reactions other than electrostatic interactions in advance. In this case, the addition of the cap can be performed more effectively.

The polyamine cap provided on the substance-encapsulated carbon nanohorn complex opens (the aperture portion opens) when the pH of an ambient environment is acid (the pH is less than 7, for example 3 to 4). As a result, the substance encapsulated in the carbon nanohorns is released to the exterior of the carbon nanohorns through the aperture portion of the carbon nanohorns. If the cap has not completely been desorbed from the carbon nanohorns, the polyamine cap can be closed (the aperture portion can be closed) so as to stop the release of the encapsulated substance by increasing the ambient pH (for example, to not less than 7) in the middle of the release.

Thus, a substance-encapsulated carbon nanohorn complex according to the present embodiment has a polyamine cap, which allows carbon nanohorns having apertures formed by oxidation to open and close the cap selectively according to a pH environment. Accordingly, it can be applied to a drug delivery system (DDS) or the like, which can control (for example, sustain) the release of an encapsulated substance such as drug.

In fact, the physiological environment in a body has a pH of 7.4. The interior of digestive organs in cells is acid. In consideration of those facts, it is possible to design a nanohorn carrier that releases an active ingredient therein when the ambient pH is lowered.

Furthermore, after nanohorn carriers have been taken in individual cancer cells through endocytosis in a local tumor, they can selectively release an internal medicine by responding under a low pH environment (pH 5) in a lysosome. Thus, nanohorn carriers can have a targeting function.

Some examples will be shown below to exemplify the present invention in greater detail. As a matter of course, the present invention is not limited to the following examples

### EXAMPLE 1

(Formation Process of Apertures in Carbon Nanohorns)

Carbon nanohorns were subject to a heat treatment at 570° C. to 580° C. under an oxygen gas atmosphere for 10 minutes. At that time, the flow rate of oxygen was set to be 200 ml/min.

(Introduction of Fullerene to the Carbon Nanohorns)

The obtained carbon nanohorns having apertures formed by oxidation (30 mg) were dispersed in toluene (40 ml). Meanwhile, fullerene (C₆₀) was used as a substance to be encapsulated in the carbon nanohorns having apertures formed by oxidation. This C₆₀ (10 mg) was immersed in the carbon nanohorns/toluene dispersion and sufficiently agitated. Then the toluene solvent was gradually evaporated and dried under a nitrogen atmosphere to produce a carbon nanohorn complex that has encapsulated C₆₀ therein. The obtained sample was subject to thermogravimetric analysis (TGA) in pure oxygen at temperatures ranging from a room temperature to 1000° C., and the amount of C₆₀ was estimated based on a difference of the combustion temperatures of C₆₀ and the carbon nanohorns,

(Production of Polyamine Cap)

The carbon nanohorn complex encapsulating C₆₀ therein (20 mg) and spermine (20 mg), which is a kind of polyamines, were dispersed in THF (tetrahydrofuran) (15 ml), which hardly dissolves C₆₀, and agitated for about 24 hours. Then filtration was performed with a filter to remove spermine that had been dissolved in THF and spermine that had not firmly been adsorbed on the carbon nanohorns. The C₆₀-encapsulated carbon nanohorn complex having a spermine cap that had remained on the filter was sufficiently dried in an inert gas. This sample was subject to thermogravimetric analysis under a helium atmosphere at temperatures ranging from a room temperature to 600° C. Under these conditions, none of C₆₀ and the carbon nanohorns is sublimated or decomposed. Accordingly, the amount of spermine adsorbed can be measured. As a result, it was seen that 30% of the total weight was adsorbed.

(Selective Release of C₆₀ by pH Change)

The release characteristics of C₆₀ encapsulated in the carbon nanohorn complex were obtained by measuring the absorption of C₆₀ that had eluted into the solution with a visible-ultraviolet absorption spectrum and converting the obtained absorption intensity of C₆₀ to the concentration of C₆₀ in the solution. For example, this experimental method is described in J. Phys. Chem. B 109, 17861 (2005).

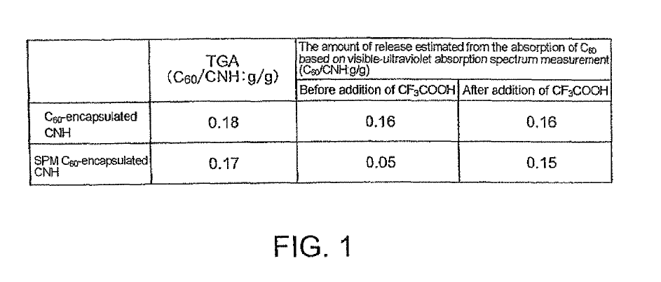

In a comparative example, a C₆₀-encapsulated carbon nanohorn complex without a cap was immersed in a toluene solution. Then C₆₀ was abruptly released from the interior of the C₆₀-encapsulated carbon nanohorn complex. The amount of C₆₀ released for approximately two hours was substantially equal to the amount of C₆₀ encapsulated which was obtained by thermogravimetric analysis (TGA) and became stable (“C₆₀-encapsulated CNH in FIG. 1”).

In contrast to the above, in the case of a C₆₀-encapsulated carbon nanohorn complex having a spermine cap, the amount of C₆₀ released for several minutes after immersion in a toluene solution reached about 30% of the amount of C₆₀ encapsulated and hardly changed thereafter. However, when the acidity was increased by gradually adding trifluoroacetic acid (CF₃COOH) to the toluene solution, the amount of C₆₀ released increased and finally became equal to the amount of C₆₀ released in the case of no cap (“SPM C₆₀-encapsulated CNH” in FIG. 1). This shows that spermine was selectively desorbed by an increase of the acidity in a solution and that C₆₀ remaining in the carbon nanohorn complex was released. It is considered that this happened because the spermine cap opened and closed according to the ambient acidity.

### EXAMPLE 2

(Formation Process of Apertures in Carbon Nanohorns)

Carbon nanohorns were subject to a heat treatment at 570° C. to 580° under an oxygen gas atmosphere for 10 minutes. At that time, the flow rate of oxygen was set to be 200 ml/min.

(Introduction of Cisplatin (CDDP: Anticancer Drug) to the Carbon Nanohorns)

The carbon nanohorns having apertures formed by oxidation (40 mg) were dispersed in N,N-dimethylformamide (DMF) (20 ml). Then CDDP to be encapsulated in the carbon nanohorns having apertures formed by oxidation was immersed in the carbon nanohorns/DMF dispersion liquid and sufficiently agitated. Thereafter, the DMF solvent was gradually evaporated and dried under a nitrogen atmosphere to produce a carbon nanohorn complex that has encapsulated CDDP therein. The obtained sample was subject to thermogravimetric analysis (TGA) in pure oxygen at temperatures ranging from a room temperature to 1000° C. and the amount of CDDP adsorbed was estimated based on the amount of residues after combustion.

(Production of Polyamine Cap)

The carbon nanohorn complex encapsulating CDDP therein (20 mg) and 1,4,8,11-tetramethyl-1,4,8,11-tetraazacyclotetradecane (TMTACTD) (20 mg), which is a kind of polyamines, were dispersed in hexane (15 ml), which hardly dissolves CDDP, and agitated for about 24 hours. Then filtration was performed with a filter, and the CDDP-encapsulated carbon nanohorn complex having a TMTACTD cap that had remained on the filter was sufficiently dried in an inert gas. This sample was subject to thermogravimetric analysis under an oxygen atmosphere at temperatures ranging from a room temperature to 1000° C.

(Selective Release of CDDP by pH Change)

As with C₆₀, the release characteristics of CDDP encapsulated in the carbon nanohorn complex were obtained by measuring the absorption of CDDP that had eluted into the solution with a visible-ultraviolet absorption spectrum and converting the measurement results to the concentration of CDDP.

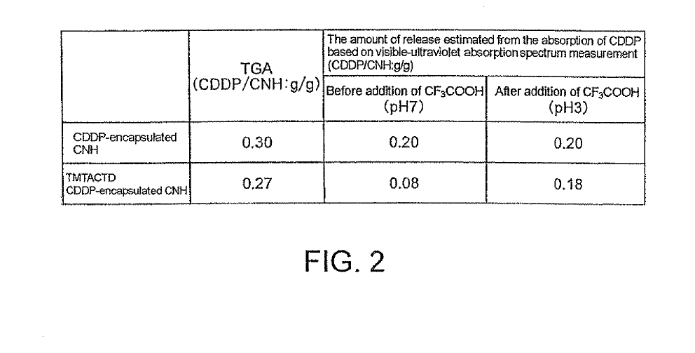

In a comparative example, a CDDP-encapsulated carbon nanohorn complex without a cap was immersed in a physiological saline solution having a pH of 7. CDDP was gradually released from the interior of the carbon nanohorns and saturated in about 40 hours. The amount of CDDP released reached 70% of the amount of encapsulation which was obtained by TGA (“CDDP-encapsulated CNH” in FIG. 2).

In contrast to the above, in the case of a CDDP-encapsulated carbon nanohorn complex having a cap of TMTACTD, the amount of CDDP released reached about 30% of the amount of CDDP encapsulated in about 50 hours after the carbon nanohorn complex had been immersed in a physiological saline solution, and CDDP was then saturated. Thereafter, when CF₃COOH was gradually added to the solution to increase the acidity from pH 7 to about pH 3, the amount of CDDP released increased and finally became equal to the amount of CDDP released in the case of no cap (“TMTACTDCDDP-encapsulated CNH” in FIG. 2). At that time, a decrease of the release rate of CDDP was seen when the acidity of the solution was set at a pH of 3, a portion of CDDP was released from the CDDP-encapsulated carbon nanohorns, and the acidity of the solution was returned to a pH of 7.

1. A substance-encapsulated carbon nanohorn complex characterized in that a cap of polyamine molecules is provided on an aperture portion of carbon nanohorns produced by an oxidation treatment so as to selectively open and close the cap according to a pH environment.

2. The substance-encapsulated carbon nanohorn complex as recited in claim 1, characterized in that an amino group of the polyamine molecules is adsorbed on a carboxyl group existing as a substituent at the aperture portion.

3. The substance-encapsulated carbon nanohorn complex as recited in claim 1, characterized in that an encapsulation substance is one of organic matter, inorganic matter, and metal, or a mixture or a compound of two or more thereof.

4. A method of producing a substance-encapsulated carbon nanohorn complex, characterized by encapsulating, in a solution, an encapsulation substance into carbon nanohorns having apertures formed by oxidation, and then attaching a cap of polyamine molecules in a solution that does not dissolve or is unlikely to dissolve the encapsulation substance so as to prevent the encapsulation substance from being released from an interior of the carbon nanohorns when the cap is being attached.

5. The method of producing a substance-encapsulated carbon nanohorn complex as recited in claim 4, characterized by cleaning the carbon nanohorns having apertures formed by oxidation after reaction with amines so as to complete an unnecessary reaction other than an electrostatic interaction in advance, and then encapsulating the encapsulation substance into the carbon nanohorns.

6. The substance-encapsulated carbon nanohorn complex as recited in claim 1, characterized in that the encapsulation substance encapsulated in the carbon nanohorns is eluted from an interior of the carbon nanohorns to an ambient environment so as to sustain release of the encapsulation substance when the cap of polyamine molecules opens.

7. A substance release control method using the substance-encapsulated carbon nanohorn complex as recited in claim 6, characterized in that a pH of an ambient environment is set to be less than 7 so as to open the cap of polyamine molecules for eluting the substance encapsulated in the carbon nanohorns to the ambient environment and sustaining release of the substance.

8. The substance release control method as recited in claim 7, characterized in that a pH of the ambient environment is set to be not less than 7 so as to close the cap of polyamine molecules for stopping the elution of the substance encapsulated in the carbon nanohorns to the ambient environment.

9. A drug delivery system (DDS) medicine characterized by including the substance-encapsulated carbon nanohorn complex as recited in claim 1.

10. The substance-encapsulated carbon nanohorn complex produced by the production method as recited in claim 4, characterized in that the encapsulation substance encapsulated in the carbon nanohorns is eluted from an interior of the carbon nanohorns to an ambient environment so as to sustain release of the encapsulation substance when the cap of polyamine molecules opens.

11. A substance release control method using the substance-encapsulated carbon nanohorn complex as recited in claim 10, characterized in that a pH of an ambient environment is set to be less than 7 so as to open the cap of polyamine molecules for eluting the substance encapsulated in the carbon nanohorns to the ambient environment and sustaining release of the substance.

12. The substance release control method as recited in claim 11, characterized in that a pH of the ambient environment is set to be not less than 7 so as to close the cap of polyamine molecules for stopping the elution of the substance encapsulated in the carbon nanohorns to the ambient environment.

13. A drug delivery system (DDS) medicine characterized by including the substance-encapsulated carbon nanohorn complex produced by the production method as recited in claim 4.
